(19) United States
(12) Patent Application Publication
(10) Pub. No.: US 2025/0154122 A1
(43) Pub. Date: May 15, 2025

(54) PROCESS FOR THE PREPARATION OF COMPOUNDS COMPRISING A 2,5-DIHYDROBENZOXEPINE STRUCTURE BY PHOTOCHEMICAL REARRANGEMENT

(71) Applicants: ECOLE POLYTECHNIQUE, Palaiseau cedex (FR); CENTRE NATIONAL DE LA RECHERCHE SCIENTIFIQUE, Paris (FR); SORBONNE UNIVERSITE, Paris (FR); ECOLE NATIONALE SUPERIEURE DE TECHNIQUES AVANCEES, Palaiseau cedex (FR)

(72) Inventors: Bastien NAY, Paris (FR); Bruce WALTERS, Orsay (FR); Simon Michel THUILLIER

(73) Assignees: ECOLE POLYTECHNIQUE, Palaiseau cedex (FR); CENTRE NATIONAL DE LA RECHERCHE SCIENTIFIQUE, Paris (FR); SORBONNE UNIVERSITE, Paris (FR); ECOLE NATIONALE SUPERIEURE DE TECHNIQUES AVANCEES, Palaiseau cedex (FR)

(21) Appl. No.: 18/839,047

(22) PCT Filed: Feb. 15, 2023
(86) PCT No.: PCT/EP2023/053766

§ 371 Date: Aug. 16, 2024

(30) Foreign Application Priority Data
Feb. 17, 2022 (FR) FR2201388

(51) Int. Cl. C07D 313/08

(52) U.S. Cl. CPC C07D 313/08

(57) ABSTRACT

The synthesis of chemical compounds having a dihydrobenzoxepine structure. In particular, a process for preparing chemical compounds having a 2,5-dihydro-benzoxepine structure by photochemical rearrangement from chemical compounds having a chromene structure. The process including a step of irradiating with ultraviolet radiation a solution in a solvent of the chemical compounds having a chromene structure.

### FIELD

The present invention applies to the field of the synthesis of chemical compounds having a dihydrobenzoxepine structure. In particular, the invention is relating to a process for preparing chemical compounds including a 2,5-dihydro-1-benzoxepine structure by photochemical rearrangement from chemical compounds having a chromene structure. This process particularly applies to the preparation of dihydrobenzoxepine derivatives with biological activity, and especially herbicidal activity, from the corresponding chromenes.

### BACKGROUND

Benzoxepine or benzooxepine is a bicyclic heterocyclic compound consisting of a benzene ring fused with that of oxepine. There are three isomers which can be represented by the following formulae:

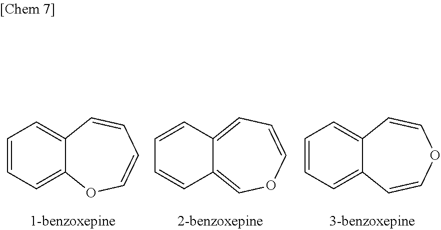

Chemical compounds with a dihydrobenzoxepine structure, also known as dihydrobenzoxepine derivatives, are compounds in which one double bond of the oxepine ring is hydrogenated.

Benzoxepine derivatives often exhibit biological activity and therefore constitute a family of compounds of interest for a variety of applications, especially in the pharmaceutical field—this is the case, for example, with doxepine, which is an anti-depressant—or in the phytopharmaceutical field—this is the case with radulanines A and H, which have herbicidal activity. In particular, bibenzyl derivatives with a 2,5-dihydro-1-benzoxepine ring include a structure of the following formula:

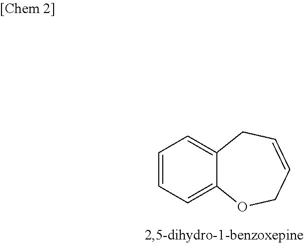 [SMILES: C1=CCc2ccccc2OC1]

Natural dihydrobenzoxepines can be isolated from plant mosses known as liverworts. These are allelopathic compounds known for their herbicidal activity. Among such compounds, mention may in particular be made of the benzoxepine derivatives described in patent application FR 3 094 869, especially including radulanine A of the following formula:

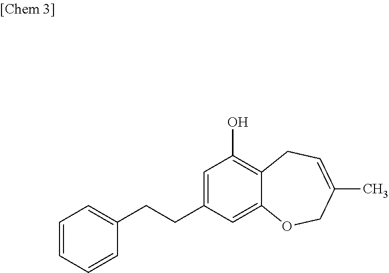 [SMILES: CC1=CCc2c(O)cc(CCc3ccccc3)cc2OC1]

Several synthetic routes leading to compounds including a 2,5-dihydrobenzoxepine structure have already been provided in the literature.

The first synthetic route uses a cyclising olefin metathesis reaction catalysed by a ruthenium complex for fusing the double bond of the 2,5-dihydrooxepine ring (M. Yoshida et al., Tetrahedron, 2009, 65, 5702-5708), according to the following scheme:

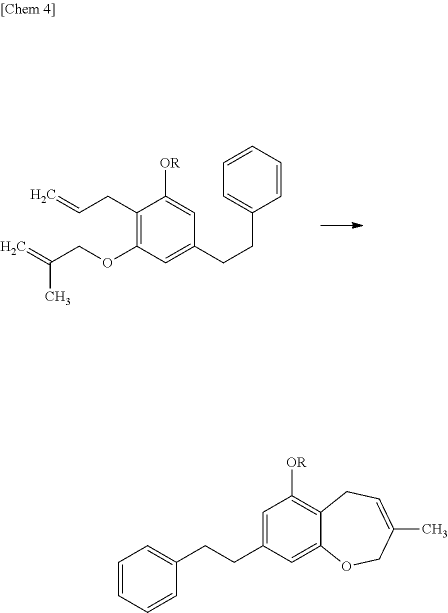

A second synthetic route uses a Mitsunobu reaction to form the cycloether linkage (S. Yamaguchi et al., Tetrahedron Letters, 2000, 41, 4787-4790) according to the following scheme:

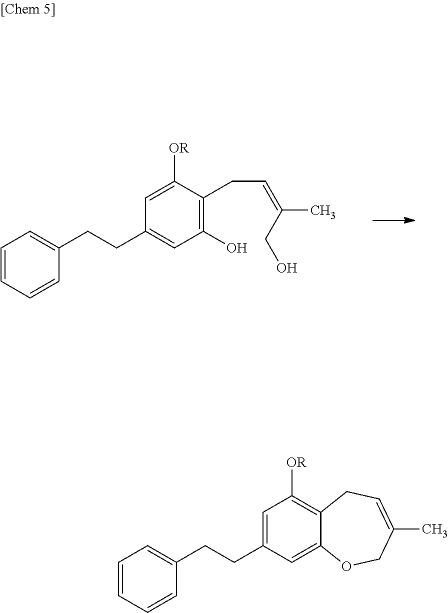

A third synthetic route uses a retro-Claisen-type rearrangement reaction starting from a vinylcyclopropane precursor (W. Zhang et al., Chem. Eur. J., 2019, 25, 8643-8648), followed by aromatisation, according to the following scheme:

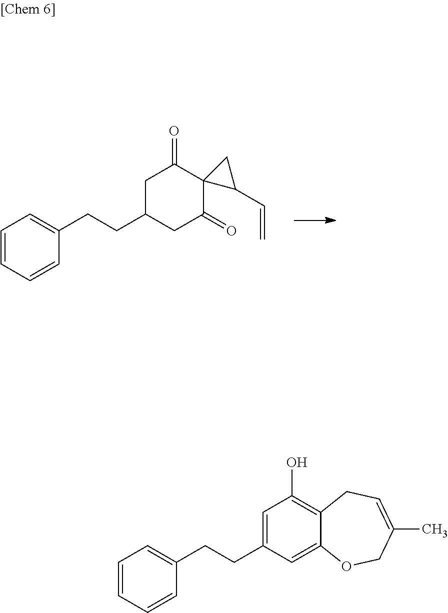 [SMILES: C=CC1CC12C(=O)CC(CCc1ccccc1)CC2=O ; CC1=CCc2c(O)cc(CCc3ccccc3)cc2OC1]

These synthetic routes take a long time to implement because obtaining radulanine precursors generally requires numerous steps implementing reactions that are tricky to carry out, either because of the sensitivity of the reagents involved, or because of the use of expensive metals such as ruthenium used as a catalyst. None of these reactions can therefore be used to synthesise radulanine, or more generally compounds including a 2,5-dihydro-1-benzoxepine structure, efficiently, rapidly and economically.

There is therefore a need for a process that provides economical access, i.e. in a few steps and without implementing expensive metals, to chemical compounds with a 2,5-dihydrobenzoxepine structure.

### SUMMARY

Thus, the aim of the present invention is to overcome drawbacks of the aforementioned prior art and to provide a process for synthesising chemical compounds having a 2,5-dihydrobenzoxepine structure.

The object of the present invention is thus a process for synthesising a compound of the following formula (I):

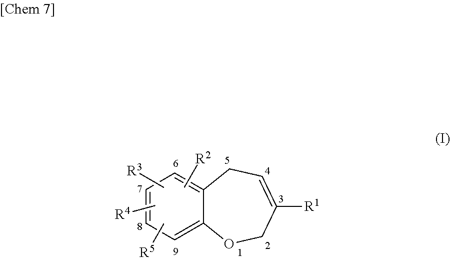

wherein:
R¹ represents a hydrogen atom or a C₁-C₆ alkyl radical,
R², R³ and R⁴, independently of each other, represent a hydrogen atom, a halogen atom, a C₁ to C₅ alkyl or cycloalkyl radical, or a group selected from —OH, —COOH, —COOR⁶, —OR⁶ and —SO₂R⁶, with R⁶ being a C₁ to C₅ alkyl or cycloalkyl radical, it being possible for said C₁ to C₅ alkyl or cycloalkyl radical to be substituted with one or more substituents selected from a halogen atom and a hydroxyl group;
R⁵ represents a hydrogen atom, a halogen atom, a C₁ to C₅ alkyl or cycloalkyl radical, or a group selected from —OH, —COOH, —COOR⁶, —OR⁶ and —SO₂R⁶, with R⁶ being a C₁ to C₅ alkyl or cycloalkyl radical, it being possible for said C₁ to C₅ alkyl or cycloalkyl radical to be substituted with one or more substituents selected from a halogen atom and a hydroxyl group, or alternatively R⁵ represents a group-L-A wherein:
L represents a bonding arm selected from linear and branched alkylene chains having at least one carbon atom, it being possible for said linear or branched alkylene chains to be interrupted and/or terminated by one or more heteroatoms selected from an oxygen, sulphur or substituted nitrogen atom, and
A represents an aromatic group selected from phenyl, naphthyl, furyl, thiophenyl, pyrrolyl, pyridinyl, indolyl, isoindolyl, benzofuryl, benzothiophenyl, quinolyl and isoquinolyl, imidazolyl, oxazolyl, thiazolyl, pyrimidyl, pyridazyl, pyrazyl, pyrrazolyl and triazolyl, it being possible for said aromatic group A to be substituted with one or more substituents selected from a halogen atom, a C₁ to C₅ alkyl or cycloalkyl radical, an —OH group, a —COOH group, a —COOR⁷ group, an —OR′ group, and an —SO₂R⁷ group, with R⁷ being a C₁ to C₅ alkyl or cycloalkyl radical, it being possible for said C₁ to C₅ alkyl or cycloalkyl radical to be substituted with one or more substituents selected from a halogen atom and a hydroxyl group, or one of the organic and inorganic salts thereof, and wherein
at least one of the groups R², R³, R⁴ and R⁵ represents an —OH group, characterised in that said process comprises a step of irradiating with ultraviolet radiation a solution in a solvent of a compound of the following formula (II):

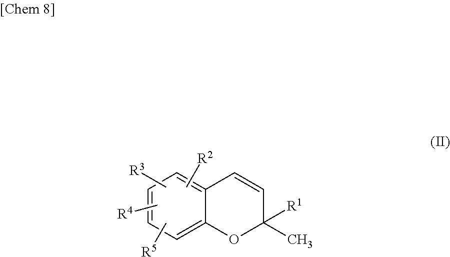

wherein R¹, R², R³, R⁴ and R⁵ have the same meaning as indicated above for the compounds of the formula (I).

The process in accordance with the present invention thus implements a ring extension reaction of a compound including a 2-methylchromene structure, preferably 2-alkyl-2-methylchromene, and most preferably 2,2-dimethylchromene, said reaction being conducted under photochemical conditions. Chromene precursors can be synthesised in a few steps from phenols and α,β-unsaturated aldehydes using acid catalysis.

In formulae (I) and (II) above, the alkyl radical indicated for R¹ may be linear or branched. It is preferably selected from group comprising a methyl radical, an ethyl radical and a t-butyl radical, the methyl radical being particularly preferred.

By way of example of inorganic salts of the compound of the formula (I), mention may be made of alkali metal and alkaline earth metal salts of the compound of the formula (I).

By way of example of organic salts of the compound of the formula (I), mention may be made of ammonium salts of the compound of the formula (I).

In the present invention, the C₁ to C₅ alkyl or cycloalkyl radical may be linear or branched, and is preferably linear.

For the purposes of the present invention, a halogen is selected from F, Cl, Br and I, and preferably from F and Cl.

Definition of R², R³, R⁴ and R⁵

The alkyl or cycloalkyl radical as the group R², R³, R⁴ or R⁵ is preferably an alkyl radical, particularly preferably a linear alkyl radical, and more particularly preferably a linear C₁ to C₃ alkyl radical.

The alkyl or cycloalkyl radical as the group R⁶ is preferably an alkyl radical, in particular a linear alkyl radical, and more particularly a linear C₁ to C₃ alkyl radical.

Said alkyl or cycloalkyl radical as the group R², R³, R⁴ or R⁵ may be substituted with one or more substituents selected from a halogen atom and a hydroxyl group.

According to a particularly preferred embodiment of the invention, at least one of the groups R², R³, R⁴ and R⁵ represents an —OH group. In this case, said at least one hydroxyl group is preferably in position 6.

In this embodiment, two of the other groups R², R³, or R² and R⁴ or R³ and R⁴ represent a hydrogen atom and R⁵ represents a group -L-A.

### Definition of L

L preferably represents a linear or branched alkylene chain having from 1 to 6 carbon atoms, particularly preferably a linear alkylene chain having from 2 to 3 carbon atoms, and more particularly preferably a linear alkylene chain having 2 carbon atoms.

The linear or branched alkylene chain as the bonding arm L may be interrupted and/or terminated by one or more heteroatoms selected from an oxygen, sulphur and substituted nitrogen atom, and preferably by one or more oxygen atoms.

The nitrogen may be substituted with a C₁ to C₅, preferably C₁ to C₃, alkyl group, said alkyl radical preferably being a linear alkyl radical.

### Definition of A

The alkyl or cycloalkyl radical as the substituent of group A is preferably an alkyl radical, particularly preferably a linear alkyl radical, and more particularly a linear C₁ to C₃ alkyl radical.

The alkyl or cycloalkyl radical as the group R⁷ is preferably an alkyl radical, in particular a linear alkyl radical, and more particularly a linear C₁ to C₃ alkyl radical.

Said alkyl or cycloalkyl radical as the substituent of group A or group R⁷ may be substituted with one or more substituents selected from a halogen atom and a hydroxyl group.

A preferably represents an aromatic group selected from phenyl, naphthyl and pyridinyl groups, and particularly preferably is a phenyl group.

The process of the invention can be carried out in static mode or in continuous flux.

### DETAILED DESCRIPTION

According to a first embodiment, the process is carried out in static mode. This first embodiment makes it possible to obtain conversion rates of the compound of the formula (II) of 90 to 100%, with yields of compound of the formula (I) in the order of 10 to 50%.

According to this first embodiment, the process according to the invention is carried out in an immersion well equipped with a tube transparent to UV radiation, for example made of Pyrex, immersed and cooled with ice water and containing a UV lamp.

According to a second embodiment, the process is carried out in continuous flux. This second embodiment makes it possible to improve speed of the reaction, reproducibility and yields of compounds including a structure of the formula (I). In this case the conversion rate of the starting compound of the formula (II) can range from 90 to 100% and the yield of compound including a structure of the formula (I) is in the order of about 30 to 50%. This second embodiment is preferred.

According to this second embodiment, the process is carried out in a continuous flux reactor consisting of a tube transparent to UV radiation, for example made of a thermoplastic material such as a perfluoroalkoxy alkane (PFA). This tube is wound around a UV lamp fitted with a Pyrex-type filter. A degassed solution of the compound of the formula (II) is injected into this reactor, in a continuous flux at a flow rate which can especially range from 0.5 to 2 mL/min.

The solvent for the solution may be selected from aromatic hydrocarbons such as benzene and toluene, acetonitrile and ethyl acetate. Among such solvents, acetonitrile and ethyl acetate are preferred, acetonitrile being particularly preferred.

The duration of the irradiation step is generally about 5 minutes to 5 hours.

According to the first embodiment of the invention, the process is carried out in static mode and the duration of the irradiation step is from 1 to 5 hours, preferably from 1 to 2 hours.

According to the second embodiment of the invention, the process is carried out in continuous flux and the duration of the irradiation step is from 5 to 20 minutes, preferably from 8 to 12 minutes.

According to the invention, by ultraviolet radiation, it is meant any invisible radiation which emits in the wavelength range from 100 to 400 nanometres (nm).

According to a preferred embodiment of the invention, the irradiation step is carried out at a wavelength of about 200 to 400 nm, and even more preferably approximately 250 to 350 nm.

The UV radiation can conventionally be generated by an ultraviolet (UV) emitting lamp. According to the invention, a medium-pressure mercury UV lamp with a power of about 100 to 400 W, preferably in the order 150 W, is preferably used.

According to the invention, and by definition, “medium pressure”, it is meant a pressure in the order of 1.105 to 1.10⁶ Pascal.

According to a particularly preferred embodiment of the invention, the process is implemented for the preparation of a compound of the following formula (Ia):

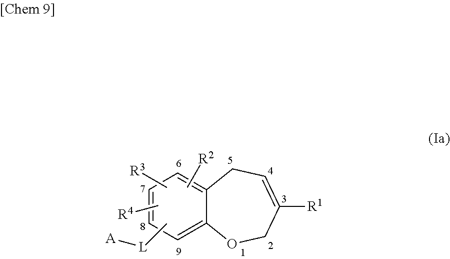

wherein R¹, R², R³, R⁴, A and L have the same meaning as indicated above for compounds of the formula (I). In this case, said compound of the formula (II) subjected to said irradiation step has the following formula (IIa):

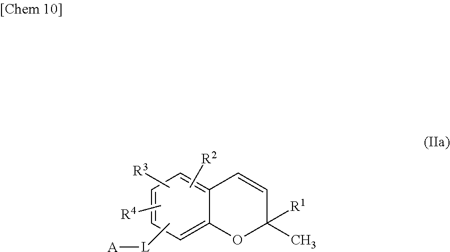

wherein R¹, R², R³, R⁴, A and L have the same meaning as indicated below for compounds of the formula (I).

Preferably, in the compounds of the formula (Ia), at least one of the groups R², R³ and R⁴ represents an —OH group. In this case, said at least one hydroxyl group is preferably in position 6. Also particularly preferably, the group -L-A is in position 8, L represents an ethylene chain and A is a phenyl ring.

According to a particularly preferred embodiment of the invention, the process is implemented for the preparation of radulanine A of the following formula (Ia-1):

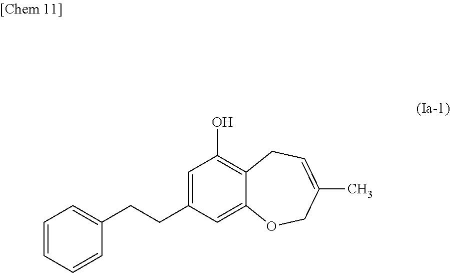 [SMILES: CC1=CCc2c(O)cc(CCc3ccccc3)cc2OC1]

Radulanine A thus corresponds to a compound of the formula (Ia) wherein R¹ represents a methyl radical, one of the groups R², R³ and R⁴ represents an OH group in position 6, the other two groups R² and R³, respectively R³ and R⁴, represent a hydrogen atom and R⁵ is a group -L-A is in position 8, and A is a phenyl ring.

Several synthetic methods can be used to afford chromenes of the formula (II). Such methods are in particular described by R. Pratap et al, Chem. Rev, 2014, 114, 10476-10526.

In particular, when the process is implemented for the preparation of a compound of the formula (I) wherein R¹ and R⁵ are as defined in formula (I), one of the groups R², R³ and R⁴ represents an OH group in position 6, the other two groups R² and R³, respectively R³ and R⁴ represent a hydrogen atom (compounds of the formula (I′), then the corresponding chromenes of the formula (II) (chromenes of the formula (II′) are preferably obtained by condensation of a diphenol of the formula (III) wherein R⁵ has the same meaning as in the formula (I) and of a α,β-unsaturated aldehyde of the formula (IV) wherein R¹ has the same meaning as in the formula (I), according to the following reaction scheme:

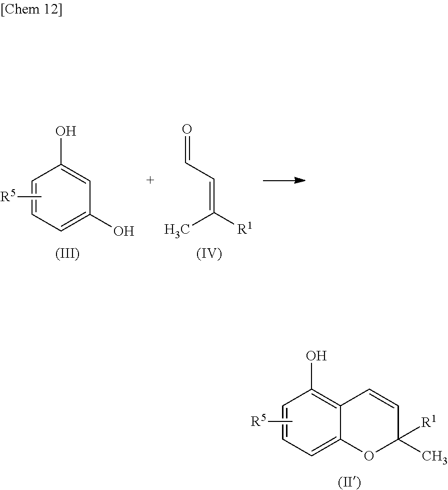

in the presence of an acid catalyst such as ethylenediammonium diacetate (EDDA) according to the method described by Lee et al (Tetrahedron Lett. 2005, 46, 7539-7543), or a Lewis acid such as Yb(OTf)₃, ZnCl₂, or a Bronsted acid such as ammonium acetate (NH₄OAc), trifluoroacetic acid (TFA), or acetic acid (AcOH); a solvent, in a closed reactor (e.g. sealed tube), under reflux, and under an inert atmosphere.

When the precursors of the formulae (111) and (IV) are not commercially available, they can be synthesised according to conventional methods. For example, unsaturated aldehydes can be obtained by a Horner-Wadsworth-Emmons type olefination reaction on a carbonyl derivative with triethyl phosphonoacetate, followed by reduction of the ester function to aldehyde. The phenolic derivatives can be obtained by various methods of electrophilic aromatic substitution well known to those skilled in the art, or by cross-coupling of activated derivatives, or even by functionalising a group already present on the aromatic system.

The reaction solvent can be selected from toluene, xylene, benzene, dichloromethane and acetic acid.

Further characteristics, alternatives and advantages of the process according to the invention will become clearer upon reading the following exemplary embodiments, which are given by way of illustrating and not limiting purposes of the invention.

### Examples

Toluene, acetonitrile and benzene have been distilled over calcium hydride before use and, if necessary, degassed by bubbling with nitrogen gas.

Analytical thin-layer chromatography (TLC) has been performed on silica gel on aluminium plates (silica gel 60, F254, Merck) and viewed by exposure to ultraviolet light and/or exposure to a basic potassium permanganate solution or a p-anisaldehyde staining solution followed by heating.

Flash column chromatography has been performed on silica 60 (40-63 pm).

Nuclear magnetic resonance spectra (¹H NMR and ¹³C NMR) have been recorded at 25° C. with a Bruker Avance 400 spectrometer (400 MHz, ¹H at 400 MHz, ¹³C NMR at 100 MHz) using CDCl₃ as the solvent referenced to residual CHCl₃ (δH=7.26 ppm, δC=77.1 ppm). Chemical shifts are given in ppm and coupling constants (J) in Hertz. Data for ¹H NMR spectra are reported as follows: chemical shift ppm (br s=broad singlet, s=singlet, d=doublet, t=triplet, q=quadruplet, dd=doublet of doublets, td=triplet of doublets, ddd=doublet of doublets of doublets, m=multiplet, coupling constants, integration).

Infrared spectra have been recorded on a PerkinElmer FTIR spectrometer using the Attenuated Total Reflectance (ATR) technique. Absorption maxima (vₘₐₓ) are reported in wave numbers (cm⁻¹).

High resolution mass spectra (HRMS) have been obtained on a JEOL JMS-GCmate II spectrometer and reported in m/z.

Batch photochemical experiments in static mode have been performed in a 500 mL immersion well or 10 mL Pyrex sealed tubes irradiated with a 150 W medium pressure Hg Heraeus lamp.

The flux photochemical experiments have been carried out on a Vapourtec E series system equipped with a UV-150 photoreactor fitted with a medium pressure Hg lamp (75-150 W) used in combination with a Pyrex filter.

### Example 1: Synthesis of Radulanine A (Compound of the Formula Ia-1) According to the Process in Accordance with the Present Invention

Radulanine A has been prepared according to a process in accordance with the present invention implementing a photochemical rearrangement step in continuous flux according to the steps illustrated in the following scheme:

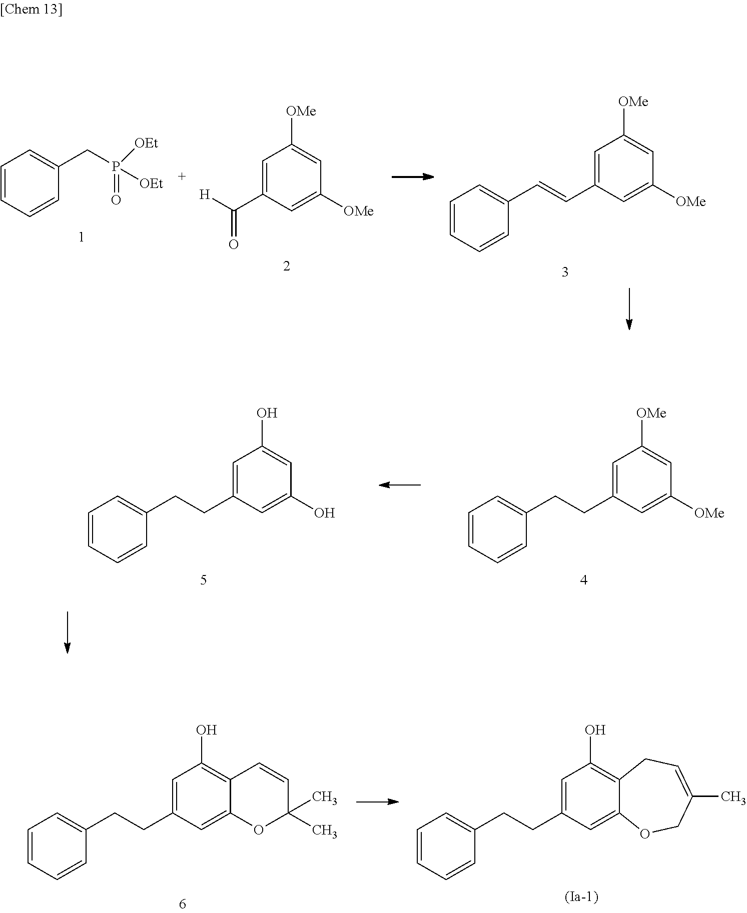

1.1 First Step: Preparation of (E)-3,5-dimethoxystilbene (Compound 3)

The first step is a Horner-Wadsworth-Emmons reaction. Potassium tert-butylate (t-BuOK) (10.8 g, 96.3 mmol) and anhydrous tetrahydrofuran (THE) (120 mL) have been added to a flame-dried 500 mL flask fitted with a magnetic stirring bar under an inert atmosphere. The mixture has been cooled in an ice bath, then diethyl benzyl phosphonate (compound 1) (20.6 mL, 90.3 mmol) has been added dropwise for 30 minutes, followed by a portionwise addition of 3,5-dimethoxybenzaldehyde (compound 2) (10.0 g, 60.2 mmol). The mixture has been allowed to rise to room temperature and then stirred for 2 h. THE has been removed in vacuo, and then a mixture of water and methanol (H₂O:MeOH) (2:1, about 60 mL) has been added until the product precipitated. Filtration and vacuum drying yielded (E)-3,5-dimethoxystilbene (compound 3) as a white solid (13.5 g, 56.0 mmol, 93% yield).

¹H NMR (400 MHz, CDCl₃): δ=7.53-7.49 (m, 2H), 7.39-7.33 (m, 2H), 7.29-7.23 (m, 1H), 7.09 (d, J=16. 3 Hz, 1H), 7.04 (d, J=16.3 Hz, 1H), 6.69-6.66 (m, 2H), 6.40 (t, J=2.3 Hz, 1H), 3.83 (s, 6H).

1.2 Second Step: Preparation of 1,3-dimethoxy-5-phenethylbenzene (Compound 4)

The second step is a catalytic hydrogenation reaction of the double bond implementing ammonium formate. It thus avoids the use of hydrogen gas. (E)-3,5-dimethoxystilbene as prepared in the previous step (14.0 g, 58.2 mmol) and 10% Pd/C (1.40 g, 10% by weight), followed by ethyl acetate (243 mL, 0.245 M) have been added to a flame-dried 500 mL flask. Ammonium formate (18.4 g, 291 mmol) has then been added and the mixture left to stir overnight at room temperature. The reaction mixture has then been filtered through celite pad and evaporated in vacuo. The remaining ammonium formate has been precipitated by the addition of dichloromethane and the mixture has been filtered again and evaporated in vacuo to give the expected 1,3-dimethoxy-5-phenethylbenzene (compound 4) as a light yellow oil (12.7 g, 52.4 mmol, 90% yield).

1 HNMR (400 MHz, CDC₁3): b=7.32-7.25 (m, 2H), 7.23-7.17 (m, 3H), 6.36-6.30 (m, 3H), 3.76 (s, 6H), 2.95-2.82 (m, 4H).

1.3. Third Step: Preparation of dihydropinosylvine (Compound 5)

The third step is a phenol demethylation in an acidic aqueous medium. To a 250 mL flask fitted with a magnetic stirring bar, 1,3-dimethoxy-5-phenethylbenzene as prepared in the previous step (2.03 g, 8.38 mmol) followed by hydrobromic acid (HBr) (24.6 mL, 48 wt % in water) and glacial acetic acid (24.6 mL, HBr:AcOH 1:1 v/v, final concentration 0.15 M) have been added. The reaction mixture has then been heated under reflux for 4 h and allowed to cool to room temperature. The reaction mixture has been diluted with water (50 mL) and extracted with diethyl ether (Et₂O) (3×50 mL). The organic phase has been treated with activated charcoal, filtered and reduced in vacuo to give dihydropinosylvine as a white solid (1.68 g, 7.86 mmol, 94%).

¹H NMR (400 MHz, CDCl₃): δ=7.33-7.25 (m, 2H), 7.24-7.15 (m, 3H), 6.31-6.18 (m, 3H), 4.71 (br s, 2H), 2.93-2.75 (m, 4H).

1.4 Fourth Step: Preparation of 2,2-dimethyl-7-phenethyl-2H-chromen-5-ol (Compound 6)

To a sealed flame-dried tube fitted with a magnetic stirring bar under an inert atmosphere the dihydropinosylvine obtained in the previous step (4.00 g, 18.7 mmoles-1 equiv.) followed by anhydrous toluene (0.1 M) and 3-methyl-2-butenal (prenal) (1.5 equiv.) have been added. Ethylenediammonium diacetate (EDDA, 5 mol %) has then been added. The container has been sealed and heated to 115° C. for 1 h. This procedure (EDDA addition and heating) has been repeated 3 times (addition of 15 mol % EDDA in total), then after returning to room temperature a small amount of silica has been added and the solvent removed under vacuum. The crude mixture has been purified by flash silica column chromatography (dry loading), eluting with hexane/ethyl acetate to give the expected 2,2-dimethyl-7-phenethyl-2H-chromen-5-ol (compound 6) as a viscous brown liquid (4.28 g, 15.3 mmol, 82% yield).

¹H NMR (400 MHz, CDCl₃): δ=7.31-7.24 (m, 2H), 7.22-7.15 (m, 3H), 6.58 (d, J=10.0, 1H), 6.32-6.29 (m, 1H), 6. 14-6.10 (m, 1H), 5.55 (d, J=10.0, 1H), 4.59 (br s, 1H), 2.92-2.83 (m, 2H), 2.80-2.73 (m, 2H), 1.42 (s, 6H).

1.5. Fifth Step: Preparation of radulanine A (Compound (Ia-1)) by Photochemical Rearrangement in Continuous Flux

To a flame-dried 1 L flask under a nitrogen atmosphere, a solution of compound 6 (200 mg, 0.713 mmol) as obtained in the previous step has been prepared in anhydrous acetonitrile degassed with nitrogen (713 mL, 0.001 M). The continuous flux system has been first rinsed with degassed anhydrous acetonitrile, then the solution of compound 6 has been injected into the photochemical reactor fitted with a Pyrex filter at 100% lamp power (150 W), at a flow rate of 1.2 mL·min⁻¹ (8.44 min residence time in the reactor), a pressure of 300 KPa and a reactor temperature of 30° C. The solution collected has been evaporated in vacuo and the crude mixture purified by flash column chromatography, eluting at 2-10% EtOAc:hexane to give radulanine A (compound of the formula (Ia-1) as a brown oil (52.2 mg, 0.186 mmol, 26%).

The NMR analysis of radulanine A is given hereinafter: ¹H NMR (400 MHz, CDCl₃): δ=7.32-7.24 and 7.22-7.14 (m, 5H), 6.53 (d, J=1.5 Hz, 1H), 6.37 (d, J=1.5 Hz, 1H), 5.64-5. 57 (m, 1H), 4.84 (br s, 1H), 4.44-4.37 (m, 2H), 3.44-3.34 (m, 2H), 2.91-2.83 and 2.83-2.75 (m, 4H), 1.57-1.50 (m, 3H).

### Example 2: Synthesis of Radulanine A (Compound of the Formula Ia-1) According to the Process in Accordance with the Invention in Static Mode

Radulanine A has been prepared according to the same steps as those illustrated in the synthetic scheme set forth above in example 1, but implementing a fifth photochemical rearrangement step in static mode.

To a sealed 10 mL flame-dried Pyrex tube fitted with a magnetic stirring bar under an inert atmosphere, the compound 5 (2.70 mg, 0.00963 mmol) as prepared above in step 4 of Example 1 and dry degassed benzene (9.00 mL, 0.001 M) have been added. The tube has been directly attached to the cooling jacket of a 150 W medium pressure mercury lamp. The reaction mixture has been irradiated for 1 hour under stirring. The solvent has been evaporated in vacuo and the crude reaction mixture subjected to ¹H NMR analysis to reveal complete conversion of compound 5 to radulanine A.

¹H NMR (400 MHz, CDCl₃): δ=7.32-7.24 and 7.22-7.14 (m, 5H), 6.53 (d, J=1.5 Hz, 1H), 6.37 (d, J=1.5 Hz, 1H), 5.64-5. 57 (m, 1H), 4.84 (br s, 1H), 4.44-4.37 (m, 2H), 3.44-3.34 (m, 2H), 2.91-2.83 and 2.83-2.75 (m, 4H), 1.57-1.50 (m, 3H).

### example 3: Synthesis of 3,8-dimethyl-2,5-dihydrobenzoxepin-6-ol (Compound of the Formula I-1) According to the Process in Accordance with the Invention in Static Mode

3,8-dimethyl-2,5-dihydrobenzoxepin-6-ol has been prepared according to a process in accordance with the present invention implementing a photochemical rearrangement step in continuous flux according to the steps illustrated in the following scheme:

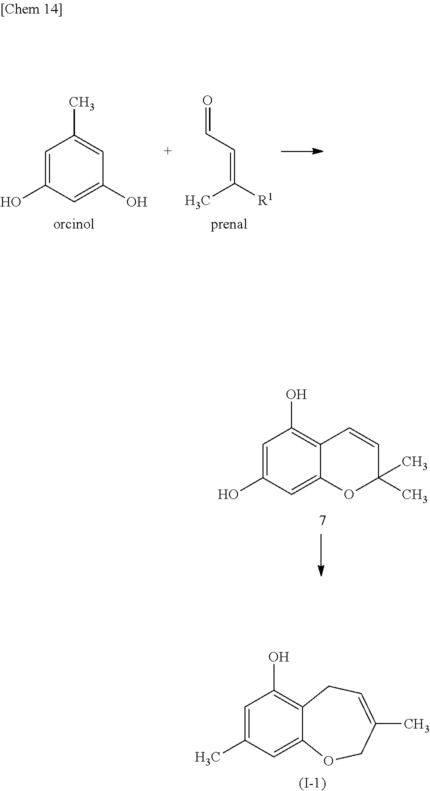

3.1 First Step: Preparation of 2,2,7-trimethyl-2H-chromen-5-ol (Compound 7)

To a sealed flame-dried tube fitted with a magnetic stirring bar under an inert atmosphere orcinol (1 equiv.) followed by anhydrous toluene (0.1 M) and prenal (1.5 equiv.) have been added. Ethylenediammonium diacetate (EDDA, 5 mol %) has then been added. The container has been sealed and heated to 115° C. for 1 h. This procedure (addition of EDDA and heating) has been repeated 3 times (addition of 15 mol % EDDA in total), then after returning to room temperature a small amount of silica has been added and the solvent removed under vacuum. The crude mixture has been purified by flash silica column chromatography (dry loading), eluting with hexane/EtOAc to give the expected 2,2,7-trimethyl-2H-chromen-5-ol (compound 7).

¹H NMR (400 MHz, CDCl₃): δ=6.57 (d, J=10.0, 1H), 6.26-6.24 (m, 1H), 6.14-6.11 (m, 1H), 5.53 (d, J=10.0 Hz, 1H), 4.60 (br s, 1H), 2.22-2.20 (m, 3H), 1.41 (s, 6H).

3.2. Second Step: Preparation of 3,8-dimethyl-2,5-dihydrobenzoxepin-6-ol (Compound of the Formula I-1)

Compound 7 obtained in the previous step (75 mg, 0.394 mmol) and anhydrous benzene degassed by bubbling with nitrogen (250 mL, 0.00158 M) have been introduced into a 500 mL immersion well equipped with a 150 W mercury lamp, a water cooling jacket and a magnetic stirring bar under an inert atmosphere. The reaction mixture has been stirred and irradiated for 30 minutes and then cooled. This procedure has been repeated 10 times until the mixture had been irradiated for a total of 5 hours. The reaction mixture has then been evaporated in a flask, under vacuum, and the crude mixture has been purified by flash column chromatography, eluting with 1:1 hexane:CH₂Cl₂, to afford the expected compound of the formula (Ia-1) as a yellow oil (35.3 mg, 0.185 mmol, 47%).

Rf=0.19 (1:1 hexane/CH₂Cl₂)

¹H NMR (400 MHz, CDCl₃): δ=6.50-6.52 (m, 1H), 6.39-6.36 (m, 1H), 5.65-5.57 (m, 1H), 4.80 (br s, 1H), 4.44-4.37 (m, 2H), 3.43-3.36 (m, 2H), 2.25-2.23 (m, 3H), 1.56-1.51 (m, 3H).

¹³C NMR (101 MHz, CDCl₃): δ=159.7, 152.0, 137.4, 134.0, 120.7, 120.1, 114.5, 112.2, 74.3, 21.6, 21.0, 20.1.

IR (ATR): 3350, 2931, 1715, 1619, 1583, 1450, 1378, 1311, 1207, 1068, 986, 836, 753.

HRMS (EI+): Calculated for C₁₂H₁₅O₂₊: 191.1067; obtained: 191.1064.

1-13. (canceled)

14. A process for synthesising a compound of the following formula (I): 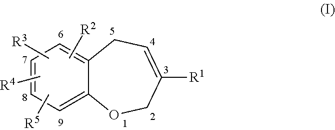
wherein:
R¹ represents a hydrogen atom or a C₁-C₆ alkyl radical,
R², R³ and R⁴, independently of each other, represent a hydrogen atom, a halogen atom, a C₁ to C₅ alkyl or cycloalkyl radical, or a group selected from —OH, —COOH, —COOR⁶, —OR⁶ and —SO₂R⁶, with R⁶ being a C₁ to C₅ alkyl or cycloalkyl radical, it being possible for said C₁ to C₅ alkyl or cycloalkyl radical to be substituted with one or more substituents selected from a halogen atom and a hydroxyl group;
R⁵ represents a hydrogen atom, a halogen atom, a C₁ to C₅ alkyl or cycloalkyl radical, or a group selected from —OH, —COOH, —COOR⁶, —OR⁶ and —SO₂R⁶, with R⁶ being a C₁ to C₅ alkyl or cycloalkyl radical, it being possible for said C₁ to C₅ alkyl or cycloalkyl radical to be substituted with one or more substituents selected from a halogen atom and a hydroxyl group, or alternatively R⁵ represents an -L-A group wherein:
L represents a bonding arm selected from linear and branched alkylene chains having at least one carbon atom, it being possible for said linear or branched alkylene chains to be interrupted and/or terminated by one or more heteroatoms selected from an oxygen, sulphur or substituted nitrogen atom, and
A represents an aromatic group selected from phenyl, naphthyl, furyl, thiophenyl, pyrrolyl, pyridinyl, indolyl, isoindolyl, benzofuryl, benzothiophenyl, quinolyl and isoquinolyl, imidazolyl, oxazolyl, thiazolyl, pyrimidyl, pyridazyl, pyrazyl, pyrrazolyl and triazolyl, it being possible for said aromatic group A to be substituted with one or more substituents selected from a halogen atom, a C₁ to C₅ alkyl or cycloalkyl radical, an —OH group, a —COOH group, a —COOR⁷ group, an —OR⁷ group, and an —SO₂R⁷ group, with R⁷ being a C₁ to C₅ alkyl or cycloalkyl radical, it being possible for said C₁ to C₅ alkyl or cycloalkyl radical to be substituted with one or more substituents selected from a halogen atom and a hydroxyl group, or one of the organic and inorganic salts thereof; and
wherein at least one of the groups R², R³, R⁴ and R⁵ represents an —OH group,
said process comprising a step of irradiating with ultraviolet radiation a solution in a solvent of a compound of the following formula (II): 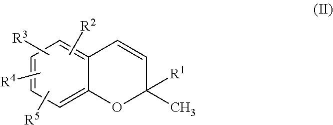
wherein R¹, R², R³, and R⁵ have the same meaning as indicated below for compounds of the formula (I).

15. The process according to claim 14, wherein the solvent of the solution is selected from aromatic hydrocarbons, acetonitrile and ethyl acetate.

16. The process according to claim 14, wherein the process is carried out in static mode and that the duration of the irradiation step is from 1 to 5 hours.

17. The process according to claim 14, wherein the process is carried out in continuous flux.

18. The process according to claim 17, wherein the duration of the irradiation step is from 5 to 20 minutes.

19. The process according to claim 14, wherein the irradiation step is carried out at a wavelength of 250 to 350 nm.

20. The process according to claim 14, wherein said at least one hydroxyl group is in position 6.

21. The process according to claim 14, wherein two of the other groups R², R³ or R² and R⁴ or R³ and R⁴ represent a hydrogen atom and R⁵ represents a group L-A.

22. The process according to claim 14, wherein L represents a linear alkylene chain having from 2 to 3 carbon atoms.

23. The process according to claim 14, wherein A represents an aromatic group selected from phenyl, thiophenyl and pyridinyl groups.

24. The process according to claim 14, wherein said process is implemented for the preparation of a compound of the following formula (Ia): 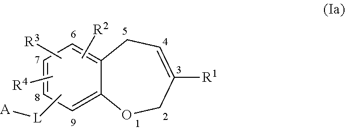
wherein R¹, R², R³, R⁴, A and L have the same meaning as indicated for the compounds of the formula (I), and
wherein said compound of the formula (II) subjected to said irradiation step corresponds to the following formula (IIa): 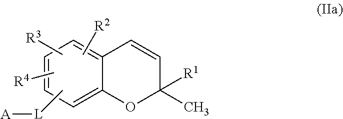
wherein R¹, R², R³, R⁴, A and L have the same meaning as indicated for the compounds of the formula (I).

25. The process according to claim 24, wherein the group L-A is in position 8, L represents an ethylene chain and A is a phenyl ring.

26. The process according to claim 24, wherein the process is implemented for the preparation of radulanine A of the following formula (Ia-1): 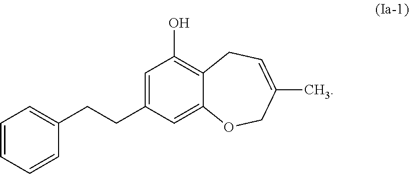 [SMILES: CC1=CCc2c(O)cc(CCc3ccccc3)cc2OC1]
